(19) United States
(12) Patent Application Publication
(10) Pub. No.: US 2009/0104211 A1
(43) Pub. Date: Apr. 23, 2009

(54) Treatment of atherosclerosis

(75) Inventors: Frank Mattner, Wien (AT); Walter Schmidt, Wien (AT)

Correspondence Address: OBLON, SPIVAK, MCCLELLAND MAIER & NEUSTADT, P.C., 1940 DUKE STREET, ALEXANDRIA, VA, 22314, US

(73) Assignee: AFFIRIS Forschungs-und Entwicklungs GmbH, Wien (AT)

(21) Appl. No.: 11/662,627

(22) PCT Filed: Sep. 8, 2005
(86) PCT No.: PCT/EP05/54445

§ 371 Date: Mar. 13, 2007

(30) Foreign Application Priority Data
Sep. 13, 2004 (AT) A 1531/2004

(51) Int. Cl. A61K 39/00; A61K 38/08; A61P 9/10; C40B 30/04

(52) U.S. Cl. 424/184.1; 514/16; 514/14; 514/15; 514/17; 506/9

(57) ABSTRACT

The invention relates to a method and a device for commissioning articles from a first number of pick-up sections (16) into a corresponding number of order placement sections (18), by means of a second number of commissioners (20). Thus, only one commissioner (20) carries out commissioning in a commissioning zone (14), such that the number of commissioning zones (14) is the same as the second number of commissioners (20) and the commissioning computer (28) varies the boundaries (15) between adjacent commissioning zones (14) by means of a zone allocation strategy (Z) in order to match the size of the commissioning zones in the commissioning region (12) and/or the number of commissioning zones (14) in the commissioning region (12) to variable influence commissioning parameters.

The invention relates to the prevention and treatment of atherosclerosis, atherosclerosis risk diseases and atherosclerosis sequelae.

Atherosclerotic sequelae, such as the peripheral arterial occlusion disease, coronary heart disease as well as the apoplectic cerebral insultus, are still among the main causes of death in the United States, Europe, and in large parts of Asia. In Virchow's view, the lipid deposits in the arterial wall were changes caused by blood lipids which he thought to be created by a transduction of lipids and complex formation with acidic mucopolysaccharides. By this “injury” of the arteries, he explains the accumulation of lipids and the development of atherosclerotic lesions in the intima and media of the arteries. Today's generally acknowledged state of knowledge is the “response to injury” hypothesis developed by Ross in 1973, and modified in 1986 and 1993. Ross considers the development of the atherosclerosis to be a chronic progressive inflammation of the arterial vessel wall which is characterized by a complex interaction of growth factors, cytokines and cell interactions. Moreover, the hypothesis also represents the integration of Virchow's lipid hypothesis with the incrustation theory of Rokitanskys. According to the “response-to-injury” hypothesis, the “injury” of the endothelium constitutes the initial event of the disease, leading to an endothelial dysfunction which triggers a cascade of cellular interactions culminating in the formation of the atherosclerotic lesions. As risk factors promoting such an “injury”, exogenous and endogenous influences are mentioned which correlate statistically significantly with atherosclerosis. Increased and modified LDL, Lp(a), arterial hypertension, Diabetes mellitus and hyperhomocysteinaemia are, for instance, counted among the most important ones of these endothelium-damaging factors. Since the endothelium does not constitute a rigid, but much rather an extremely dynamic barrier, a plurality of molecular changes occur in the course of the endothelial dysfunction in addition to an increased permeability for lipoproteins, which molecular changes have a decisive influence on the interaction of monocytes, T-lymphocytes and endothelial cells. By the expression of endothelial adhesion molecules of the type of the E, L and P selectins, integrins, ICMA-1, VCAM-1 and platelet-endothelial-cell adhesion molecule-1, adhesion of monocytes and T-lymphocytes at the lumen side occurs. The subsequent migration of the leukocytes over the endothelium is mediated by MCP-1, interleukin-8, PDGF, M-CSF and osteopontin. Via the so-called scavenger receptor, macrophages and monocytes resident in the intima are capable of taking up the penetrated LDL particles and to deposit them as vacuoles of cholesterol esters in the cytoplasma. The foam cells formed in this manner accumulate mainly in groups in the region of the vessel intima and form the “fatty streak” lesions occurring already in childhood. LDL are lipoproteins of low density and are formed by catabolic effects of lipolytic enzymes from VLDL particles rich in triglyceride. Besides their damaging properties on endothelial cells and smooth muscle cells of the media, LDL moreover has a chemotactic effect on monocytes and is capable of increasing the expression of MCSF and MCP-1 of the endothelial cells via gene amplification. In contrast to LDL, HDL is capable of taking up cholesterol esters from loaded macrophages mediated by apolipoprotein E, under formation of so-called HDLc complexes. By the interaction of SR-B1 receptors, these cholesterol ester-loaded particles are capable of binding to hepatocytes or to cells of the adrenal cortex and delivering cholesterol for the production of bile acids and steroids, respectively. This mechanism is called reverse cholesterol transport and elucidates the protective function of HDL. Activated macrophages are capable of presenting antigens via HLA-DR and thereby activate CD4 and CD8 lymphocytes which, consequently, are stimulated to secrete cytokines, such as IFN-gamma and TNF-alpha, and moreover, contribute to increasing the inflammatory reaction. In the further course of the disease, smooth muscle cells of the media start to grow into the region of the intima which has been altered by inflammation. By this, the intermediary lesion forms at this stage. Starting from the intermediary lesion, the progressive and complicated lesion will develop over time, which is morphologically characterized by a necrotic core, cellular detritus and a fibrinous cap rich in collagen on the side of the lumen. If the cell number and the portion of the lipoids increase continuously, tears in the endothelium will occur, and surfaces with thrombotic properties will be exposed. Due to the adhesion and activation of thrombocytes at these tears, granules will be released which contain cytokines, growth factors and thrombin. Proteolytic enzymes of the macrophages are responsible for the thinning of the fibrinous cap which, at last, will lead to a rupture of the plaques with consecutive thrombosis and stenosing of the vessels and an acute ischemia of the terminal vessels.

Various risk factors are held responsible for the forming of atherosclerotic lesions. Hyperlipoproteinemia, arterial hypertension and abuse of nicotine are of particular significance in this respect. A disease which involves an excessive increase in the total and LDL cholesterol is the familial hypercholesterinemia. It belongs to the most frequent monogenetically inherited metabolic diseases. The moderate heterozygous form occurs with a frequency of 1:500, the homozygous form with 1:1 million clearly more rarely. Causes of the familial hypercholesterinemia are mutations in the LDL receptor gene on the short arm of chromosome 19. These mutations may be deletions, insertions or point mutations. The characteristic finding of the lipoproteins in familial hypercholesterinemia is an increase in the total and LDL cholesterol at mostly normal triglyceride and VLDL concentrations. Often the HDL is lowered. Phenotypically, there is a type IIAa-hyperlipoproteinemia according to Fredrikson. In the heterozygous form, the total cholesterol is increased by the two to three-fold, in the homozygous form it is increased by the five to six-fold as compared to the normal level. Clinically the familial hypercholesterinemia manifests itself by an early coronary sclerosis. As a rule, in heterozygous men the first symptoms of a coronary heart disease (CHD) occur between their 30^{th} and the 40^{th} year of age, in women on an average 10 years later. 50% of the afflicted men die of the consequences of their coronary sclerosis before they are 50 years old. Besides the massively increased LDL levels, also lowered HDL concentrations are responsible for the rapid progress of atherosclerosis. Atherosclerotic changes may become manifest also on extracardiac vessels, such as the aorta, the carotid arteries and peripheral arteries. With the homozygous form of the disease, the coronary sclerosis develops already in early childhood. The first myocardial infarction often occurs before the 10^{th} year of age, and in most cases the afflicted persons die before they are 20 years old. The development of xanthomas is a function of the level of the serum cholesterol and the duration of the disease. Approximately 75% of the heterozygous individuals afflicted who are more than 20 years old exhibit tendinous xanthomas. The homozygous individuals have skin and tendon xanthomas in nearly 100%. Lipid deposits may also occur on the eye lid and in the cornea (xanthelasmas; Arcus lipoides). These are, however, not a specific sign of a hypercholesterinemia, since they are also found with normal cholesterol levels. Furthermore, with the FH, acute arthritides and tendosynovitides occur frequently. The individual lipoproteins differ with respect to size and density, since they contain differently large portions of lipids and proteins, so-called apoproteins. The density increases with increasing protein and decreasing lipid portion. Due to their different densities, they can be separated into different fractions by ultracentrifugation. This is the basis for the classification of the lipoproteins into their main groups: chylomicrones, very-low-density lipoproteins (VLDL), intermediate-density lipoproteins (IDL), low-density lipoproteins (LDL), high-density lipoproteins (HDL), lipoprotein (a) (Lp(a)). Among the lipoproteins with a high atherogenic potential there are primarily the LDL, the Lp(a) and the VLDL. LDL has a density of approximately d=1.006-1.063 g/ml. The core is formed by esterified cholesterol molecules. This highly hydrophobic core is surrounded by an envelope of phospholipids, non-esterified cholesterol and one single Apo B100 molecule. Besides, Apoprotein E is found on the surface of the LDL particles. The function of the LDL consists in transporting cholesterol to peripheral tissues where—mediated by the apoprotein B-100—it is taken up into the cells via the LDL receptor. In comprehensive epidemiologic studies, such as the Framingham Study, the Multiple Risk Factor Intervention Trial and the Whitehall Study, a positive correlation between the level of the serum cholesterol and the occurrence of a coronary heart disease could be demonstrated. LDL cholesterol levels of higher than 160 mg/dl constitute a high cardiovascular risk. Besides the level of the LDL cholesterol, also the level of the vessel-protecting HDL cholesterol plays an important role when estimating the risk profile for cardiovascular diseases. Levels of below 35 mg/dl are associated with an increased risk. VLDL are lipoproteins with a low density (d=0.94-1.006 g/ml) and a high triglyceride portion. Substantially, VLDL contain apoprotein C, and small portions of apoproteins B-100 and E. Different from chylomicrons, VLDL do not consist of food lipids, but are synthesized in the liver from endogenously formed triglycerides and secreted into circulation. As with the chylomicrons, the triglycerides are hydrolyzed by the apoprotein C-II-activated lipoprotein-lipase, and the free fatty acids are supplied to the muscle and fat tissue. The remaining cholesterol-rich VLDL remnants are called intermediate density lipoproteins because of their higher density. Lipoprotein(a) (Lp(a)) has a density of 1.05 to 1.12 g/ml and resembles LDL in its composition. Besides apoprotein B-100, its protein portion consists of the apoprotein(a) which is characteristic of Lp(a). To date, very little is known about the physiology and function of the Lp(a). Since the apoprotein(a) molecule has a high sequence homology to plasminogen, it is assumed that Lp(a) both promotes the formation of thrombi on atherosclerotic plaques and also has an atherogenic effect. Lp(a) is found together with apoprotein B in atherosclerotic lesions. Retrospective studies have shown a correlation between increased Lp(a) and a CHD. Likewise, the metaanalysis of numerous prospective studies has shown that Lp(a) is an independent risk factor for the occurrence of a CHD. Levels of between 15 and 35 mg/dl are considered to be normal. So far, Lp(a) can be influenced neither by diet nor by medicaments. Therefore, therapy measures are restricted to reducing further risk factors. In particular, a lowering of the LDL cholesterol seems to lower the cardiovascular risk of Lp(a). In the pathogenesis of atherosclerosis, considerable pathophysiologic importance is, moreover, attributed to coagulation factors. Epidemiologic findings suggest a correlation between the fibrinogen concentration in plasma and the development of a coronary heart disease, and, primarily, a myocardial infarction. In this context, increased fibrinogen levels (>300 mg/dl) proved to be an independent indicator and risk factor for cardiovascular diseases. Yet also high concentrations of the tissue plasminogen activator inhibitor tPA-I are associated with the occurrence of CHD. The relationship between hyper-triglyceridemia and coronary risk is a different one in each case, depending on the cause of the elevation of the blood lipids. Despite the discussion whether or not triglycerides are to be considered as an independent risk factor it is undisputed that they play an important role in the pathogenesis of coronary heart diseases. Incidence of the disease is the highest in patients who exhibit high LDL cholesterol and a high triglyceride level.

The cholesterol ester transfer protein (CETP) is a stable plasma glycoprotein which is responsible for the transfer of neutral lipids and phospholipids between lipoproteins and which down-regulates the plasma concentration of HDL. The inhibition of the CETP lipid transfer activity has already been suggested as a therapeutic approach for increasing the HDL plasma level. There are numerous reasons which suggest that the absence of CETP activity in plasma should lead to an increase in the HDL levels. Thus, CETP lowers the HDL concentration by the transfer of cholesterol esters from HDL to LDL and VLDL. In animal experiments with rabbits and hamsters, the transient inhibition of CETP with anti-CETP monoclonal antibodies, antisense oligonucleotides or CETP inhibitors led to the increase in the HDL levels. Lasting CETP inhibition with antisense oligonucleotides increased the HDL levels and, thus, led to a reduction of the atherosclerotic lesions in the rabbit animal model for atherosclerosis. With the heterozygous gene defect, patients with familial hypercholesterolemia have CETP plasma levels twice as high as those of healthy humans, with the homozygous gene defect, the levels are even three times as high.

In U.S. Pat. No. 5,512,548 and in WO 93/011782, polypeptides and their analogues are described which are capable of inhibiting CETP that catalyses the transfer of cholesterol esters from HDL to VLDL and LDL, and, therefore, have anti-atherosclerotic activity if administered to a patient. According to these documents, such a CETP polypeptide inhibitor is derived from apolipoprotein C-I of various sources, wherein especially N-terminal fragments up to amino acid 36 have been identified as CETP inhibitors.

Also in U.S. Pat. No. 5,880,095 A, a CETP-binding peptide is disclosed which is capable of inhibiting the activity of CETP in an individual. The CETP-inhibitory protein comprises an N-terminal fragment of porcine apolipoprotein C-III.

In US 2004/0087481 and U.S. Pat. No. 6,410,022 B1, peptides are disclosed which, because of the induction of a CETP-specific immune response, can be used for the treatment and prevention of cardiovascular diseases, such as, e.g., atherosclerosis. These peptides comprise a T helper cell epitope which is not derived from CETP, and at least one B-cell epitope that comes from CETP and can be derived directly from the latter. The T helper cell epitope advantageously is derived from tetanus toxoid and is covalently bound to at least one B-cell epitope of CETP. By using a T helper cell epitope that is alien to the organism, it becomes possible to induce antibodies in the body of an individual, which antibodies are directed against that peptide portion that consists of at least one CETP-B-cell epitope.

Most recently, there have already been suggestions for a vaccine approach with regard to CETP. Thus, e.g., rabbits have been treated with a vaccine which contained that peptide of CETP responsible for the cholesterol-ester transfer as an antigen. The immunized rabbits had a reduced CETP activity and altered lipoprotein levels with increased HDL and reduced LDL values. Moreover, the treated test animals of the atherosclerosis model also showed reduced atherosclerotic lesions in comparison with control animals.

At the end of last year, the results of a phase II-clinical study were published, which study had been carried out by the American biotechnology company Avant with the vaccine CETi-1 (BioCentury Extra For Wednesday, Oct. 22, 2003). In this phase II-study, just as in the preceding phase I-study, a very good safety profile without any questionable side effects was proven, allowing the conclusion to be drawn that basically no side effects are to be expected from an anti-CETP vaccination approach. With regard to efficacy, however, the Avant vaccine was disappointing since it did not lead to increased HDL levels significantly better than those attained by a placebo treatment.

The problem with the CETi-1 vaccine is that it uses endogenous antigen. The human immune system is tolerant relative to endogenous structures, since with most of the endogenous molecules—other than with CETP—it is vital that no autoantibodies be formed. Thus, it was the object of the CETi-1 vaccine to break the endogenous tolerance which, apparently, it has not achieved to a sufficient extent.

Thus, it is the object of the present invention to provide an antigen for an anti-CETP vaccine which is selected such that it is considered as foreign by the immune system and therefore need not break a self-tolerance.

Therefore, the present invention provides a CETP mimotope for these purposes. The CETP mimotopes according to the present invention preferably are antigenic polypeptides which in their amino acid sequence are completely different from the amino acid sequence of CETP or of fragments of CETP. In this respect, the inventive mimotope may comprise one or more non-natural amino acids (i.e. not from the 20 “classical” amino acids) or it may be completely assembled of such non-natural amino acids. Moreover, the inventive antigens which induce anti-CETP antibodies may be assembled of D or L amino acids or of combinations of DL amino acids and, optionally, may have been changed by further modifications, ring closures or derivatizations. Suitable anti-CETP-antibody-inducing antigens may be provided from commercially available peptide libraries. Preferably, these peptides are at least 5 amino acids in length, in particular at least 8 amino acids, and preferred lengths may be up to 11, preferably up to 14 or 20 amino acids. According to the invention, however, also longer peptides may very well be employed as anti-CETP-antibody-inducing antigens.

For preparing such CETP-mimotopes (i.e. anti-CETP-antibody-inducing antigens), of course also phage libraries, peptide libraries are suitable, for instance produced by means of combinatorial chemistry or obtained by means of high throughput screening techniques for the most varying structures (Display: A Laboratory Manual by Carlos F. Barbas (Editor), et al.; Willats W G Phage display: practicalities and prospects. Plant Mol. Biol. 2002 December; 50(6):837-54). (http://www.microcollections.de/showpublications.php#).

Furthermore, according to the invention also anti-CETP-antibody-inducing antigens based on nucleic acids (“aptamers”) may be employed, and these, too, may be found with the most varying (oligonucleotide) libraries (e.g. with 2-180 nucleic acid residues) (e.g. Burgstaller et al., Curr. Opin. Drug Discov. Dev. 5(5) (2002), 690-700; Famulok et al., Acc. Chem. Res. 33 (2000), 591-599; Mayer et al., PNAS 98 (2001), 4961-4965, etc.). In anti-CETP-antibody-inducing antigens based on nucleic acids, the nucleic acid backbone can be provided e.g. by the natural phosphor-diester compounds, or also by phosphorothioates or combinations or chemical variations (e.g. as PNA), wherein as bases, according to the invention primarily U, T, A, C, G, H and mC can be employed. The 2′-residues of the nucleotides which can be used according to the present invention preferably are H, OH, F, Cl, NH₂, O-methyl, O-ethyl, O-propyl or O-butyl, wherein the nucleic acids may also be differently modified, i.e. for instance with protective groups, as they are commonly employed in oligonucleotide synthesis. Thus, aptamer-based anti-CETP-antibody-inducing antigens are also preferred anti-CETP-antibody-inducing antigens within the scope of the present invention.

According to a further aspect, the present invention relates to the use of a compound comprising the following amino acid sequence

X₁X₂X₃X₄X₅X₆X₇X₈,

wherein
X₁ is an amino acid other than C,
X₂ is an amino acid other than C,
X₃ is an amino acid other than C,
X₄ is an amino acid other than C,
X₅ is an amino acid other than C,
X₆ is not present or is an amino acid other than C,
X₇ is not present or is an amino acid other than C,
X₈ is not present or is an amino acid other than C,
and wherein X₁X₂X₃X₄X₅X₆X₇X₈ is not, or does not comprise, a 5-mer, 6-mer, 7-mer or 8-mer polypeptide fragment of the cholesterol ester transport protein (CETP) or a CETP-epitope, said compound having a binding capacity to an antibody which is specific for the natural CETP glycoprotein, for producing a means for pre-venting and treating atherosclerosis, atherosclerosis risk diseases and atherosclerosis sequelae.

Particularly preferred compounds are specific mimotopes for per se known CETP-epitopes, in particular for those epitopes which are defined by the amino acids 131-142, 451-476, 184-260, 261-331, 332-366, 367-409 and 410-450 of the CETP amino acid sequence, in particular FGFPEHLLVDFLQSLS or CDSGRVRTDAPD.

### Total CEPT Sequence (Unprocessed Precursor):

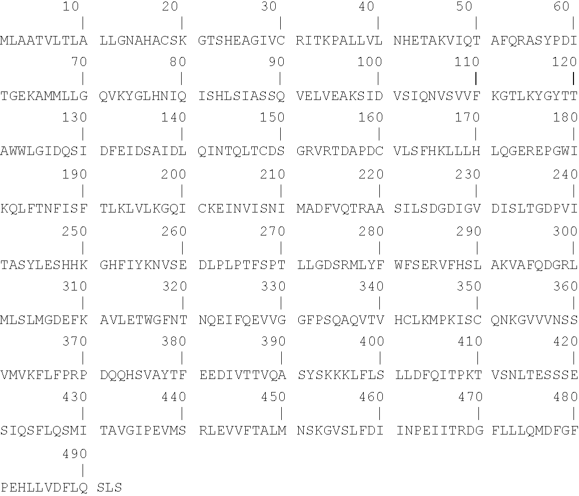

The compound according to the invention (mimotope) has a preferred length of from 5 to 15 amino acids. This compound may be provided in the vaccine in isolated (peptide) form, or it may be coupled to or complexed with other molecules, such as pharmaceutical carrier substances or polypeptide, lipid or carbohydrate structures. Preferably, the mimotopes according to the invention have a (minimum) length of between 5 and 15, 6 and 12 amino acid residues, specifically between 9 and 11. The mimotopes may, however, be (covalently or non-covalently) coupled to non-specific linkers or carriers, in particular to peptide linkers or protein carriers. Furthermore, the peptide linkers or protein carriers may consist of T cell helper epitopes or contain the same.

Preferably, the pharmaceutically acceptable carrier is KLH, tetanus toxoid, albumin-binding protein, bovine serum albumin, a dendrimer (MAP; Biol. Chem. 358: 581) as well as the adjuvant substances described in Singh et al., Nat. Biotech. 17 (1999), 1075-1081 (in particular those in Table 1 of that document), and O'Hagan et al., Nature Reviews, Drug Discovery 2 (9) (2003), 727-735 (in particular the endogenous immuno-potentiating compounds and delivery systems described therein), or mixtures thereof. Moreover, the vaccine composition may contain aluminium hydroxide.

A vaccine which comprises the present compound (mimotope) and the pharmaceutically acceptable carrier may be administered by any suitable mode of application, e.g. i.v., i.p., i.m., intranasally, orally, subcutaneously, etc. and in any suitable delivery device (O'Hagan et al., Nature Reviews, Drug Discovery 2 (9), (2003), 727-735). Typically, the vaccine contains the compound according to the invention in an amount of from 0.1 ng to 10 mg, preferably 10 ng to 1 mg, in particular 100 ng to 100 μg, or, alternatively, e.g. 100 fmol to 10 μmol, preferably 10 pmol to 1 μmol, in particular 100 pmol to 100 nmol. Typically, the vaccine may also contain auxiliary substances, e.g. buffers, stabilizers etc.

Particularly suitable for the inventive vaccine composition for the prevention and treatment of atherosclerosis, atherosclerosis risk diseases and atherosclerosis sequelae proved to be molecules which contain a peptide that has a binding capacity to an antibody which is specific for the natural CETP glycoprotein and which is encompassed by the general formula

X₁X₂X₃X₄X₅X₆X₇X₈,

wherein
X₁ is any amino acid or is not present, preferably is A, L, I or is not present, with the proviso that if X₁ is not present, X₆ is present,
X₂ is D, G, A, N, L, V, Q or I, in particular L, V, Q or I,
X₃ is H, P, K or R, in particular K or R,
X₄ is any amino acid (other than C), in particular W, N, S, G, H, Y, D or E,
X₅ is H, S, T, P, K or R, in particular K or R,
X₆ is not present or is N, F, H, L, V or I, in particular L, V or I,
X₇ is not present or is W, L, V, I, F, N, P or G, in particular P or G,
X₈ is not present or is any amino acid other than C.
These molecules preferably are peptides which comprise the general peptide sequence described here as part of a larger peptide molecule, or which consist of this molecule. Particularly preferred are here one or more peptides selected from the group ALKNKLP, ALKSKIP, AVKGKLP, ALKHKIP, ALKHKVP, ALKNKIP, ALKGKIP, ALKYKLP, ALKDKLP, ALKDKVP, AAQKDKVP, LKLHHGTPFQFN, SLPPDHWSLPVQ, QQQLGRDTFLHL or TNHWPNIQDIGG.

In peptides which are also advantageous, the above formula is defined as follows (of course, always with the proviso of the specific binding capacity to CETP/CETP fragment):

X₁ is A, L or I, in particular A,

### X₂ is L, V, Q or I,

### X₃ is K or R,

X₄ is any amino acid (other than C), in particular N, S, G, H, Y, D or E,

### X₅ is K or R,

X₆ is not present or is L, V or I,
X₇ is not present or is P or G,
X₈ is not present or is any amino acid other than C.

According to a further aspect, the present invention relates to a method of isolating a compound which binds to an antibody that is specific for natural CETP or a CETP fragment, comprising the following steps:

providing a peptide compound library comprising peptides which contain the following amino acid sequence

X₁X₂X₃X₄X₅X₆X₇X₈,

wherein
X₁ is an amino acid other than C,
X₂ is an amino acid other than C,
X₃ is an amino acid other than C,
X₄ is an amino acid other than C,
X₅ is an amino acid other than C,
X₆ is not present or is an amino acid other than C,
X₇ is not present or is an amino acid other than C,
X₈ is not present or is an amino acid other than C,
and wherein X₁X₂X₃X₄X₅X₆X₇X₈ is not, or does not comprise, a 5-mer, 6-mer, 7-mer or 8-mer polypeptide fragment of the cholesterol ester transport protein (CETP) or a CETP-epitope,

contacting said peptide library with this antibody, and

isolating those members of the peptide library which bind to this antibody.

Such a method proved to be successful for obtaining the CETP mimotopes according to the invention. Antibodies which are specific for the natural CETP or a CETP fragment have been extensively described in the prior art or commercially provided (e.g. U.S. Pat. No. 6,410,022 or 6,555,113).

Preferably, these peptides are provided in said library in individualized form, i.e. as individual peptides, in particular immobilized on a solid surface as is feasible e.g. by means of MULTIPIN™ peptide technology. The library may also be provided as a peptide mixture, and the antibody-peptide complexes may be isolated after said antibody binding. As an alternative, the antibody may be immobilized, and the peptide library (in suspension or in solution) is then contacted with the immobilized antibodies.

Preferably, the screening antibodies (or the members of the peptide library) comprise a suitable marker which enables the detection or the isolation of the antibody or of the antibody:peptide complex when binding to a peptide of the library. Suitable marker systems (i.a. biotinylation, fluorescence, radioactivity, magnetic markers, colour-developing markers, secondary antibodies) are easily available to the person skilled in the art.

The library must be constructed to exclude the naturally occurring CETP sequences, since a vaccination with this sequence is clearly excluded by this invention.

A further suitable technique for isolating the epitope according to the present invention is the screening in phage-peptide libraries as described e.g. in WO 03/020750.

The present invention also relates to a vaccine for the prevention and treatment of atherosclerosis, atherosclerosis risk diseases and atherosclerosis sequelae, comprising an antigen which contains at least one peptide selected from the group ALKNKLP, ALKSKIP, AVKGKLP, ALKHKIP, ALKHKVP, ALKNKIP, ALKGKIP, ALKYKLP, ALKDKLP, ALKDKVP, AAQKDKVP, LKLHHGTPFQFN, SLPPDHWSLPVQ, QQQLGRDTFLHL or TNHWPNIQDIGG. In addition to the other peptides provided with the present invention, these peptides are specifically suitable to be used for the production of a pharmaceutical composition, in particular for atherosclerosis vaccines. These sequences are purely artificial CETP-mimotopes. For vaccination purposes, the peptides may (covalently or non-covalently) be coupled to suitable carriers and may be provided as peptide compounds or complexes in combination with other compounds or moieties, e.g. adjuvants, peptides or protein carriers, etc., and be administered in a suitable way (such as, e.g., in O'Hagan et al., Nature Reviews, Drug Discovery 2 (9) (2003), 727-735.

Finally, the present invention also relates to the use of a CETP mimotope for producing a means for preventing and treating atherosclerosis, atherosclerosis risk diseases and atherosclerosis sequelae. In this respect, the CETP mimotope according to the invention may comprise a peptide structure (as the inventively screened library peptides) or (e.g. as aptamers) have other structures (e.g. on nucleic acid basis). It is merely essential that they have an affinity to antibodies against the natural CETP which approximately corresponds to that of the natural sequences (at least 50% of the binding affinity), yet do not contain any “self-structures”.

The invention will be explained in more detail by way of the following example without, however, being restricted thereto.

### EXAMPLE

There exists a strong inverse relationship between the plasma concentration of cholesterol in high density lipoproteins (HDLs) and the development of coronary heart disease (CHD) (1). Thus, the risk for CHD is higher when HDLs decrease. Although 33% of patients with CHD have low plasma levels of HDLs, there is currently no effective therapy for increasing the plasma concentration of HDLs. Diet and moderate exercise are ineffective (2), statins only achieve a low 5 to 7% increase in HDL (3), and niacin has side effects and compliance profiles limiting its use (4).

The inhibition of CETP activity has been suggested as therapeutic approach to increase plasma HDL levels (5). CETP is a plasma glycoprotein that facilitates transfer of neutral lipids and phospholipids between lipoproteins and regulates the concentration of plasma HDL (6). The inhibition of CETP activity is expected to increase plasma HDL concentrations for several reasons. CETP lowers HDL concentrations by moving cholesteryl esters from HDLs to VLDLs and LDLs (5). Transient inhibition of CETP in rabbits and hamsters by monoclonal antibodies (7, 8), small molecules (9), or antisense oligonucleotides (10) causes HDL increase. Sustained CETP inhibition with antisense nucleotides increased plasma HDL and reduced atherosclerotic lesions in a rabbit model of atherosclerosis (11). CETP-transgenic mice (12) and rats (13) show decreased plasma HDL. Humans with reduced CETP activity have elevated plasma HDL (14).

Recently, a vaccine approach has been proposed (15). Rabbits were immunized with a human CETP-derived peptide containing a region of CETP critical for neutral lipid transfer function. Vaccinated rabbits had reduced CETP activity and an altered lipoprotein profile with lower LDL and higher HDL concentration. Furthermore, CETP-vaccinated rabbits were shown to have smaller atherosclerotic lesions than control animals.

The problem of the anti-CETP vaccine approach discussed above is that the vaccine formulation comprises a self peptide and therefore must break natural tolerance against self antigens. The invention describes a CETP mimotope that can be used for vaccination: The mimotope shall induce the production of antibodies against CETP. The CETP mimotope does not have a self sequence and therefore does not need to break tolerance. Thus, the induction of an anti-CETP antibody response is greatly facilitated. The mimotope is identified with a monoclonal antibody (mAb) and (commercially available) peptide libraries (e.g. according to 16). An anti-CETP monoclonal antibody is used that neutralizes CETP activity (17). This mAb detects a sequence within the C-terminal 26 amino acids of CETP necessary for neutral lipid transfer activity (18).

CETP is a 476 amino acid glycoprotein. The following regions within the protein have been described to be immunogenic:

Amino acids 131-142 (19)
Amino acids 451-476 (20, 21)
Amino acids 184-260 (22)
Amino acids 261-331 (22)
Amino acids 332-366 (22)
Amino acids 367-409 (22)
Amino acids 410-450 (22)

Inhibitory as well as non-inhibitory antibodies detecting the above listed regions within CETP can be used to detect mimotopes.

The Sequences

One monoclonal antibody used for the mimotope identification detects the CETP-derived amino acid sequence FGFPEHLLVDFLQSLS (=original epitope).

The mimotope has a preferred length of 5 to 15 amino acids. Two different libraries are used in ELISA assays to define mimotope sequences.

Library 1: This 7 mer library contains peptides with the following sequences (amino acid positions 1 to 7):

Position 1: all natural aa except of C (19 possibilities)
Position 2: all natural aa except of C (19 possibilities)
Position 3: all natural aa except of C (19 possibilities)
Position 4: all natural aa except of C (19 possibilities)
Position 5: all natural aa except of C (19 possibilities)
Position 6: all natural aa except of C (19 possibilities)
Position 7: all natural aa except of C (19 possibilities)
The 7 mer peptides ALKNKLP, ALKSKIP, AVKGKLP, ALKHKIP, ALKHKVP, ALKNKIP, ALKGKIP, ALKYKLP, ALKDKLP, and ALKDKVP are examples for mimotopes detected by a monoclonal antibody.

Library 2: This 8 mer library contains peptides with the following sequences (amino acid positions 1 to 8):

Position 1: all natural aa except of C (19 possibilities)
Position 2: all natural aa except of C (19 possibilities)
Position 3: all natural aa except of C (19 possibilities)
Position 4: all natural aa except of C (19 possibilities)
Position 5: all natural aa except of C (19 possibilities)
Position 6: all natural aa except of C (19 possibilities)
Position 7: all natural aa except of C (19 possibilities)
Position 8: all natural aa except of C (19 possibilities)

The 8 mer peptide AAQKDKVP is an example for a mimotope detected by a monoclonal antibody.

Another monoclonal antibody used for the mimotope identification detects the CETP-derived amino acid sequence CDSGRVRTDAPD (=Original epitope).

The mimotope used for vaccination has to be administered in an immunogenic form, e.g. coupled to a carrier.

### REFERENCES

(1) Gordon et al 1989: “High-density lipoprotein: the clinical implications of recent studies” N Engl J Med 321:1311
(2) Stefanick et al 1998: “Effects of diets and exercise in men and postmenopausal women with low levels of HDL cholesterol and high levels of LDL cholesterol” N Engl J Med 339:12
(3) Schonfeld et al 1998: “Role of 3-hydroxy-3-methylglutaryl coenzyme A reductase inhibitors (“statins”) in familial combined hyperlipidemia” Am J Cardiol 81:43B
(4) King et al 1994: “Evaluation of effects of unmodified niacin on fasting and postprandial plasma lipids in normolipidemic men with hypoalphalipoproteinemia” Am J Med 97:323
(5) Tall 1993: “Plasma cholesteryl ester transfer protein” J Lipid Res 34:1255
(6) Barter et al 1994: “Cholesteryl ester transfer protein: its role in plasma lipid transport” Clin Exp Pharmacol Physiol
(7) Whitlock et al 1989: “Monoclonal antibody inhibition of cholesteryl ester transfer protein activity in the rabbit: effects on lipoprotein composition and high density lipoprotein cholesteryl ester metabolism” J Clin Invest 84:129
(8) Gaynor et al 1994: “Inhibition of cholesteryl ester transfer protein activity in hamsters alters HDL lipid composition” Atherosclerosis 110:101
(9) Kothari et al 1997: “Inhibition of cholesteryl ester transfer protein by CGS 25159 and changes in lipoproteins in hamsters” Atherosclerosis 128:59
(10) Sugano et al 1996: “Changes in plasma lipoprotein cholesterol levels by antisense oligodeoxynucleotides against cholesteryl ester transfer protein in cholesterol-fed rabbits” J Biol Chem 271:19080
(11) Sugano et al 1998: “Effect of antisense oligonucleotides against cholesteryl ester transfer protein on the development of atherosclerosis in cholesterol-fed rabbits” J Biol Chem 273:5033
(12) Agellon et al 1991: “Reduced high density lipoprotein cholesterol in human cholesteryl ester transfer protein transgenic mice” J Biol Chem 266:10796
(13) Herrera et al 1999: “Spontaneous combined hyperlipidemia, coronary heart disease and decreased survival Dahl-salt sensitive hypertensive rats transgenic for human cholesteryl ester transfer protein” Nat Med 5:1383
(14) Koizumi et al 1985: “Deficiency of serum cholesteryl-ester transfer protein activity in patients with familial hyperalphalipoproteinaemia” Atherosclerosis 58:175
(15) Rittershaus et al 2000: “Vaccine-induced antibodies inhibit CETP activity in vivo and reduce aortic lesions in a rabbit model of atherosclerosis” Arterioscler Thromb Vasc Biol 20:2106
(16) Reineke et al. 2002: “Identification of distinct antibody epitopes and mimotopes from a peptide array of 5520 randomly generated sequences” J Immunol Methods 267:37
(17) Swenson et al 1989: “Mechanism of cholesteryl ester transfer protein inhibition by a neutralizing monoclonal antibody and mapping of the monoclonal antibody epitope” J Biol Chem
(18) Wang et al 1992: “Identification of a sequence within the C-terminal 26 amino acids of cholesteryl ester transfer protein responsible for binding a neutralizing monoclonal antibody and necessary for neutral lipid transfer activity” J Biol Chem
(19) Thomas et al 1996: “Mouse monoclonal antipeptide antibodies specific for cholesteryl ester transfer protein (CETP).” Hybridoma 15(5):359
(20) Hesler et al 1988: “Monoclonal antibodies to the Mr 74,000 cholesteryl ester transfer protein neutralize all of the cholesteryl ester and triglyceride transfer activities in human plasma Biol Chem 258:11751
(21) Swenson et al 1989: “Mechanism of cholesteryl ester transfer protein inhibition by a neutralizing monoclonal antibody and mapping of the monoclonal antibody epitope” J Biol Chem
(22) Roy et al 1996: “Structure-function relationships of human cholesteryl ester transfer protein: analysis using monoclonal antibodies” J Lipid Res 37:22

1: A method for preventing and treating atherosclerosis, atherosclerosis risk diseases and atherosclerosis sequelae comprising administering to a subject in need thereof an effective amount of a compound comprising the following amino acid sequence
X₁X₂X₃X₄X₅X₆X₇X₈,
wherein
X₁ is an amino acid other than C,
X₂ is an amino acid other than C,
X₃ is an amino acid other than C,
X₄ is an amino acid other than C,
X₅ is an amino acid other than C,
X₆ is not present or is an amino acid other than C,
X₇ is not present or is an amino acid other than C,
X₈ is not present or is an amino acid other than C,
and wherein X₁X₂X₃X₄X₅X₆X₇X₈ is not, or does not comprise, a 5-mer, 6-mer, 7-mer or 8-mer polypeptide fragment of the cholesterol ester transport protein (CETP) or a CETP-epitope, said compound having a binding capacity to an antibody which is specific for the natural CETP glycoprotein.

2: The method according to claim 1, wherein the compound is a CETP-mimotope for a CETP-epitope selected from the group of epitopes consisting of the amino acids 131-142, 451-476, 184-260, 261-331, 332-366, 367-409 and 410-450 of the CETP-amino acid sequence.

3: The method according to claim 1, wherein the compound is a polypeptide comprising 5 to 15 amino acid residues.

4: The method according to claim 1 wherein the compound is coupled to a pharmaceutically acceptable carrier, and optionally aluminium hydroxide.

5: The method according to claim 1 wherein the compound is contained in an amount of from 0.1 ng to 10 mg.

6: The method according to claim 1 wherein the compound comprises the following peptides: ALKNKLP (SEQ ID NO: 4), ALKSKIP (SEQ ID NO: 5), AVKGKLP (SEQ ID NO: 6), ALKHKIP (SEQ ID NO: 7), ALKHKVP (SEQ ID NO: 8), ALKNKIP (SEQ ID NO: 9), ALKGKIP (SEQ ID NO: 10), ALKYKLP (SEQ ID NO: 11), ALKDKLP (SEQ ID NO: 12), ALKDKVP (SEQ ID NO: 13), AAQKDKVP (SEQ ID NO: 14), LKLHHGTPFQFN (SEQ ID NO: 15), SLPPDHWSLPVQ (SEQ ID NO: 16), QQQLGRDTFLHL (SEQ ID NO: 17) or TNHWPNIQDIGG (SEQ ID NO: 18).

7: A method for isolating a compound which binds to an antibody that is specific for natural CETP or a CETP-fragment, comprising the following steps:
providing a peptide compound library comprising peptides which contain the following amino acid sequence
X₁X₂X₃X₄X₅X₆X₇X₈,
wherein
X₁ is an amino acid other than C,
X₂ is an amino acid other than C,
X₃ is an amino acid other than C,
X₄ is an amino acid other than C,
X₅ is an amino acid other than C,
X₆ is not present or is an amino acid other than C,
X₇ is not present or is an amino acid other than C,
X₈ is not present or is an amino acid other than C,
and wherein X₁X₂X₃X₄X₅X₆X₇X₈ is not, or does not comprise, a 5-mer, 6-mer, 7-mer or 8-mer polypeptide fragment of the cholesterol ester transport protein (CETP) or a CETP-epitope,
contacting said peptide library with this antibody, and
isolating those members of the peptide library which bind to this antibody.

8: The method according to claim 7, wherein the peptides in the said library are provided in individualized form.

9: The method according to claim 7 wherein said antibody comprises a suitable marker which enables detection or isolation of said antibody when bound to a peptide of the library.

10: A vaccine for the prevention and treatment of atherosclerosis, atherosclerosis risk diseases and atherosclerosis sequelae, comprising an antigen which includes at least one peptide which has a binding capacity to an antibody that is specific for the natural CETP glycoprotein and is encompassed by the general formula
X₁X₂X₃X₄X₅X₆X₇X₈,
wherein
X1 is any amino acid or is not present, preferably is A, L, I or is not present, with the proviso that if X1 is not present, X6 is present,
X2 is D, G, A, N, L, V, Q or I, in particular L, V, Q or I,
X3 is H, P, K or R, in particular K or R,
X4 is any amino acid (other than C), in particular W, N, S, G, H, Y, D or E,
X5 is H, S, T, P, K or R, in particular K or R,
X6 is not present or is N, F, H, L, V or I, in particular L, V or I,
X7 is not present or is W, L, V, I, F, N, P or G, in particular P or G,
X8 is not present or is any amino acid other than C, in particular a peptide selected from the group of ALKNKLP (SEQ ID NO: 4), ALKSKIP (SEQ ID NO: 5), AVKGKLP (SEQ ID NO: 6), ALKHKIP (SEQ ID NO: 7), ALKHKVP (SEQ ID NO: 8), ALKNKIP (SEQ ID NO: 9), ALKGKIP (SEQ ID NO: 10), ALKYKLP (SEQ ID NO: 11), ALKDKLP (SEQ ID NO: 12), ALKDKVP (SEQ ID NO: 13), AAQKDKVP (SEQ ID NO: 14), LKLHHGTPFQFN (SEQ ID NO: 15), SLPPDHWSLPVQ (SEQ ID NO: 16), QQQLGRDTFLHL (SEQ ID NO: 17) or TNHWPNIQDIGG (SEQ ID NO: 18).

11: A method for producing a means for preventing and treating atherosclerosis, atherosclerosis risk diseases and atherosclerosis sequelae, comprising incorporating a CETP-mimotope.

12: The method according to claim 2 wherein the CETP-mimotope for a CETP-epitope is FGFPEHLLVDFLQSLS (SEQ ID NO: 1) or CDSGRVRTDAPD (SEQ ID NO: 2).

13: The method according to claim 4 wherein the pharmaceutically acceptable carrier is KLH.

14: The method according to claim 5, wherein the compound is contained in an amount of from 10 ng to 1 mg.

15: The method according to claim 5, wherein the compound is contained in an amount of from 100 ng to 10 μg.

16: The method according to claim 8 wherein said individualized form is immobilized on a solid surface.


SEQUENCE LISTING (18 sequences)

SEQ ID NO: 1 - protein, 16 aa, Homo sapiens
FGFPEHLLVDFLQSLS

SEQ ID NO: 2 - protein, 12 aa, Homo sapiens
CDSGRVRTDAPD

SEQ ID NO: 3 - protein, 493 aa, Homo sapiens
MLAATVLTLALLGNAHACSKGTSHEAGIVCRITKPALLVLNHETAKVIQTAFQRASYPDITGEKAMMLLGQVKYGLHNIQISHLSIASSQVELVEAKSIDVSIQNVSVVFKGTLKYGYTTAWWLGIDQSIDFEIDSAIDLQINTQLTCDSGRVRTDAPDCYLSFHKLLLHLQGEREPGWIKQLFTNFISFTLKLVLKGQICKEINVISNIMADFVQTRAASILSDGDIGVDISLTGDPVITASYLESHHKGHFIYKNVSEDLPLPTFSPTLLGDSRMLYFWFSERVFHSLAKVAFQDGRLMLSLMGDEFKAVLETWGFNTNQEIFQEVVGGFPSQAQVTVHCLKMPKISCQNKGVVVNSSVMVKFLFPRPDQQHSVAYTFEEDIVTTVQASYSKKKLFLSLLDFQITPKTVSNLTESSSESIQSFLQSMITAVGIPEVMSRLEVVFTALMNSKGVSLFDIINPEIITRDGFLLLQMDFGFPEHLLVDFLQSLS

SEQ ID NO: 4 - protein, 7 aa, Artificial Sequence
  Description of Artificial Sequence Synthetic peptide
ALKNKLP

SEQ ID NO: 5 - protein, 7 aa, Artificial Sequence
  Description of Artificial Sequence Synthetic peptide
ALKSKIP

SEQ ID NO: 6 - protein, 7 aa, Artificial Sequence
  Description of Artificial Sequence Synthetic peptide
AVKGKLP

SEQ ID NO: 7 - protein, 7 aa, Artificial Sequence
  Description of Artificial Sequence Synthetic peptide
ALKHKIP

SEQ ID NO: 8 - protein, 7 aa, Artificial Sequence
  Description of Artificial Sequence Synthetic peptide
ALKHKVP

SEQ ID NO: 9 - protein, 7 aa, Artificial Sequence
  Description of Artificial Sequence Synthetic peptide
ALKNKIP

SEQ ID NO: 10 - protein, 7 aa, Artificial Sequence
  Description of Artificial Sequence Synthetic peptide
ALKGKIP

SEQ ID NO: 11 - protein, 7 aa, Artificial Sequence
  Description of Artificial Sequence Synthetic peptide
ALKYKLP

SEQ ID NO: 12 - protein, 7 aa, Artificial Sequence
  Description of Artificial Sequence Synthetic peptide
ALKDKLP

SEQ ID NO: 13 - protein, 7 aa, Artificial Sequence
  Description of Artificial Sequence Synthetic peptide
ALKDKVP

SEQ ID NO: 14 - protein, 8 aa, Artificial Sequence
  Description of Artificial Sequence Synthetic peptide
AAQKDKVP

SEQ ID NO: 15 - protein, 12 aa, Artificial Sequence
  Description of Artificial Sequence Synthetic peptide
LKLHHGTPFQFN

SEQ ID NO: 16 - protein, 12 aa, Artificial Sequence
  Description of Artificial Sequence Synthetic peptide
SLPPDHWSLPVQ

SEQ ID NO: 17 - protein, 12 aa, Artificial Sequence
  Description of Artificial Sequence Synthetic peptide
QQQLGRDTFLHL

SEQ ID NO: 18 - protein, 12 aa, Artificial Sequence
  Description of Artificial Sequence Synthetic peptide
TNHWPNIQDIGG